(19) United States
(12) Patent Application Publication
(10) Pub. No.: US 2021/0093772 A1
(43) Pub. Date: Apr. 1, 2021

(54) BLOOD TREATMENT DEVICE WITH AUTOMATIC SUBSTITUTION VOLUME COMPENSATION

(71) Applicant: B. Braun Avitum AG, Melsungen (DE)

(72) Inventors: ISTVÁN GOLARITS, Budapest (HU); Peter PÓZNA, Budapest (HU); BOTOND TÉNYI, Budapest (HU)

(21) Appl. No.: 17/032,111

(22) Filed: Sep. 25, 2020

(30) Foreign Application Priority Data
Sep. 26, 2019 (DE) 10 2019 126 048.5

(51) Int. Cl. A61M 1/36; A61M 1/16

(52) U.S. Cl. CPC A61M 1/3606; A61M 1/367; A61M 1/1625; A61M 1/3633; A61M 1/3663

(57) ABSTRACT

A blood treatment device includes an extracorporeal blood circuit, a dialyzer and a dialysis fluid circuit. The extracorporeal blood circuit and the dialysis fluid circuit are separated from each other by a membrane provided in the dialyzer, by which blood can be filtered. At least one substitution solution pump supplies a substitution solution to the extracorporeal blood circuit before and/or after the dialyzer. A control unit calculates a difference or a backlog between an ideal target volume and an actually controlled volume of the supplied substitution solution, and temporarily increases a controlled flow rate of the substitution solution pump under corresponding controlling thereof by a predetermined, fixed percentage which is less than or equal to 5%, until the difference or the backlog between the actually controlled volume and the ideal target volume no longer exists, i.e. the actually controlled volume corresponds to the ideal target volume.

### CROSS-REFERENCE TO RELATED APPLICATIONS

This application claims priority under 35 U.S.C. § 119 to German Application No. 10 2019 126 048.5, filed Sep. 26, 2019, the content of which is incorporated by reference herein in its entirety.

### FIELD

The present disclosure relates to a blood treatment device, in particular a dialysis device, for use in (continuous) blood treatment/dialysis therapies, in particular renal replacement therapies, comprising: an extracorporeal blood circuit, a dialyzer and a dialysis fluid circuit, wherein the extracorporeal blood circuit and the dialysis fluid circuit are separated from each other via a membrane provided in the dialyzer, via which blood can be filtered (using a dialysis fluid solution); and at least one substitution solution pump, which is configured to supply a substitution solution to the extracorporeal blood circuit before and/or after the dialyzer.

### BACKGROUND

Blood treatment devices are already known from the prior art. For example, EP 0 321 754 A1 discloses a blood treatment device having a filter divided into two chambers by a membrane. An extracorporeal blood circuit is passed through one chamber of the filter. The other chamber of the filter is connected to an ultrafiltration unit, which is configured to withdraw effluent from the other chamber using an effluent pump. The blood treatment device comprises a substitution unit, which is configured to supply a substitution fluid to the blood circuit by means of a substitution pump. Furthermore, the blood treatment device has a scale that balances the amount of effluent removed and the amount of substitution fluid added, by weighing the effluent container and the substitution fluid container with their respective contents. Furthermore, the blood treatment device contains a control unit to control the effluent pump and the substitution pump.

Another document, EP 0 829 265 B1, also discloses a blood treatment device that comprises an interface for a disposable tubing set, a plurality of pumps such as a blood pump, a syringe pump, an effluent pump and a substitution pump, load cells for measuring the weight of bags containing fluids required for the blood treatment, a user interface comprising a display with touch screen and a control unit for controlling the processes of the blood treatment device.

Further prior art can be found in EP 0 373 455 A1, CA 2 580 848 A1, U.S. Pat. No. 5,470,483 A, WO 94/11093 A1, DE 33 13 421 A1, WO 92/00768 A1, WO 2018/017623 A1, and U.S. Pat. No. 9,089,639 B2.

During a dialysis treatment, events can basically occur which contribute to the fact that an intended ideal/optimal target substitution fluid volume/substitution solution volume set by a user cannot be achieved. For example, this can happen when the substitution solution pump/pump flow rate is started up/started/restarted at the beginning of a therapy. Moreover, errors can occur in the supply of the substitution solution, for example, if a bag containing the substitution solution is not correctly connected to a tube that supplies the substitution solution to the extracorporeal blood circuit.

In principle, the prior art has the disadvantage that deviations (i.e. differences/backlogs) between a (predetermined) ideal/optimal target volume set by a user and an actually/concretely controlled volume of the supplied substitution solution cannot be automatically compensated in a simple way.

### SUMMARY

It is therefore the object of the present disclosure to avoid or at least reduce the disadvantages of the prior art. In particular, the blood treatment device is to be configured in such a way that deviations/differences between an ideal/optimal target volume and an actually controlled volume of the supplied substitution solution or backlogs of the actually controlled volume with respect to the ideal target volume are gradually compensated over the course of a therapy, so that the ideal (desired) target volume is achieved (again).

This object is solved in a generic blood treatment device in that it has a control unit that is configured to calculate a deviation/backlog/difference between a (predetermined) ideal/optimal target volume set by a user and an actually/concretely controlled volume of the substitution solution supplied, and temporarily increasing a controlled (through-) flow rate/delivery rate/delivery amount/a controlled volume flow of the substitution solution pump under corresponding driving thereof by a predetermined, fixed percentage which is less than or equal to 5% (compared to a preset/original or normal/desired/actually required flow rate of the substitution solution pump), specifically until the deviation between the actually controlled volume and the ideal target volume no longer exists, i.e. the actually controlled volume corresponds to the ideal target volume.

In other words, the control unit of the present disclosure calculates a deviation between volumes, specifically between a target volume of the substitution solution to be supplied to the extracorporeal blood circuit and an actually supplied/controlled volume/actual volume/a quantity actually supplied/delivered. In particular a backlog/a catch-up demand of the actual volume compared to the target volume is calculated. If there is a deviation/a backlog, the flow rate of the substitution pump/the substitution solution flow rate is temporarily increased, i.e. briefly for a certain period of time. The core of the disclosure is that the percentage increase in the flow rate of the substitution pump is 5% maximum. Accordingly, the control unit sets a new, increased flow rate. As soon as there is no difference anymore between the actually controlled volume and the target volume/there is no backlog, the original/normal/preset/actually required/desired flow rate of the substitution solution pump is set again. Deviations between the ideal target volume and the actually controlled volume of the supplied substitution solution can thus be compensated automatically in a simple way according to the disclosure.

In other words, according to the present disclosure, the control unit is configured to compensate for the difference or backlog between the ideal target volume and the actually controlled volume by temporarily increasing the controlled flow rate of the at least one substitution solution pump by the predetermined, fixed percentage.

It is advantageous if the predetermined, fixed percentage by which the volume flow of the substitution solution is increased is at least 1% and at most 5%. If the percentage is between 1% and 5%, the deviation or the backlog is removed promptly, but not too quickly, so that the control unit can react in time when the actually controlled volume corresponds to the ideal target volume and can reset the flow rate of the substitution solution pump to the (actually desired) initial value. In this way, it is preferably excluded that the actually controlled volume becomes larger than the target volume during an increase of the flow rate. Furthermore, it has been found that if the percentage is greater than 1%, the actual volume flow/the actual flow rate will normally become large enough to compensate for the above mentioned events that contribute to the fact that the intended substitution fluid volume/substitution solution volume is not reached.

Preferably, the predetermined, fixed percentage is set by the control unit depending on the missing volume, so that the predetermined, fixed percentage is set higher if the deviation between the actually controlled volume and the ideal target volume is large, than if the deviation between the actually controlled volume and the ideal target volume is small. For example, the predetermined, fixed percentage is set to 1% when the deviation between the actually controlled volume and the ideal target volume is small and the predetermined, fixed percentage is set to 5% when the deviation between the actually controlled volume and the ideal target volume is large. However, any percentage increase between 1% and 5% is also possible.

Furthermore, it is advantageous if therapy stop times triggered by an alarm are taken into account in the calculation of the deviation between the actual volume and the target volume. If an alarm is triggered, a therapy is principally stopped. Therefore, no substitution solution is added to the extracorporeal blood circuit. During the alarm/therapy stop, no fluid volume has to be compensated and the control unit does not take into account a supplied/delivered quantity during the alarm/therapy stop.

It is advantageous if the control unit is configured to raise an alarm when it detects that even if the flow rate of the substitution solution pump is increased by 5%, the deviation between the actual volume and the target volume cannot be compensated.

It is advantageous if the control unit is configured to increase the flow rate of the substitution solution pump only if this is not prohibited by other restrictions/conditions.

Preferably, the control unit is configured to calculate the difference or the backlog between the ideal target volume and the actually controlled volume using the course of the flow rate (set by the control unit) of the at least one substitution solution pump.

It is practical, if the control unit is configured to set the flow rate or the volume flow of the at least one substitution solution pump.

Preferably, when (re)starting at least one substitution solution pump, the flow rate/volume flow increases slowly/continuously/linearly, so that a desired flow rate/a desired volume flow is reached only after a predetermined short time period.

In particular, the volume flow/the flow rate increases linearly from zero to the desired flow rate (volume flow) upon start/restart.

Preferably, the control unit is configured to temporarily increase the flow rate by the predetermined, fixed percentage after reaching the desired flow rate/the desired volume flow in order to slowly/continuously reduce the difference/backlog between the actually controlled volume and the ideal target volume, which results from the slow/continuous increase of the flow rate at startup/restart, specifically until the difference or the backlog between the actually controlled volume and the ideal target volume no longer exists, i.e. the actually controlled volume corresponds to the ideal target volume.

If the controlled flow rate of the substitution solution pump has to be reduced temporarily (e.g. due to a temporary blockage of the dialyzer), the control unit is advantageously configured to reduce or compensate (in retrospect) for the resulting backlog or the resulting difference between the ideal target volume and the actually controlled volume, by temporarily increasing the controlled flow rate of the substitution solution pump by the predetermined, fixed percentage under appropriate control of the same, specifically until the difference or the backlog between the actually controlled volume and the ideal target volume no longer exists, i.e. the actually controlled volume corresponds to the ideal target volume.

In the event that an actually controlled volume of the supplied substitution solution is larger than the ideal target volume set by the user, the control unit can basically also be configured to temporarily reduce the controlled flow rate of the substitution solution pump by a/the predetermined, fixed percentage, specifically until there is no deviation between the actually controlled volume and the ideal target volume, i.e. the actually controlled volume corresponds to the ideal target volume.

Preferably, the blood treatment device has a weighing device, in particular a load cell, to measure the weight of a bag, in particular a disposable bag, containing the substitution solution.

It is practical if the extracorporeal blood circuit and the dialysis fluid circuit are designed as disposable tubes, which are attached to an interface provided on the dialysis device.

In addition to the substitution solution pump, the plurality of pumps preferably includes at least one blood pump, one syringe pump and one effluent pump.

Furthermore, the blood treatment device is preferably equipped with a bar code reader, which is configured to read bar codes on disposable items such as disposable tubing or their packaging.

Moreover, the blood treatment device preferably has a user interface comprising a display with touch screen.

The blood treatment device is preferably configured for wired communication.

The control unit of the blood treatment device is preferably designed as at least one processor, preferably several processors.

In other words, the disclosure relates to a dialysis device. The dialysis device includes a bar code reader. Furthermore, the dialysis device contains a user interface or a display with a touch screen. The dialysis device also has an interface for a disposable tubing set containing a blood side and a dialysis-fluid side separated by a (semi)permeable membrane for filtering blood (using a dialysis fluid solution/dialysis solution). A substitution solution/replacement solution is supplied to the blood side before/after a dialyzer. The dialysis device has a blood pump, a syringe pump, an effluent pump, a substitution solution pump etc. The dialysis device is configured for wired communication/has wired or wire-connected communication facilities. The dialysis device is characterized by a software that is particularly suitable for use in continuous dialysis therapies, such as renal replacement therapy. The software runs on a large number of processors within the dialysis device. The dialysis device also has an energy management device (integrated circuit). The dialysis device also contains weighing devices, in particular load cells, which measure the weight of disposable bags containing the fluids (e.g. dialysis fluid solution, substitution solution) required for the dialysis therapy.

The present system or dialysis device is designed to compensate for the backlogs between the actual volume and the target volume of the substitution solution during the course of therapy, so that the target volume is ultimately reached. If the system detects a deviation between target and actual volume, the substitution fluid flow rate is temporarily increased by 1% to 5% (depending on the missing volume). When the backlog/deviation is removed, this function is switched off.

### BRIEF DESCRIPTION OF THE DRAWING FIGURES

The disclosure is further explained in the following with the help of figures. These show:

FIG. 1 shows a schematic view of a blood treatment device according to the present disclosure;

FIG. 2 shows a flow chart illustrating the automatic compensation of a volume of the substitution solution running in the control unit according to the disclosure; and

FIG. 3 shows a diagram showing a time course of a substitution solution flow rate, according to the present disclosure.

### DETAILED DESCRIPTION

The figures are merely schematic in nature and serve exclusively for understanding the present disclosure. The same elements are marked with the same reference signs.

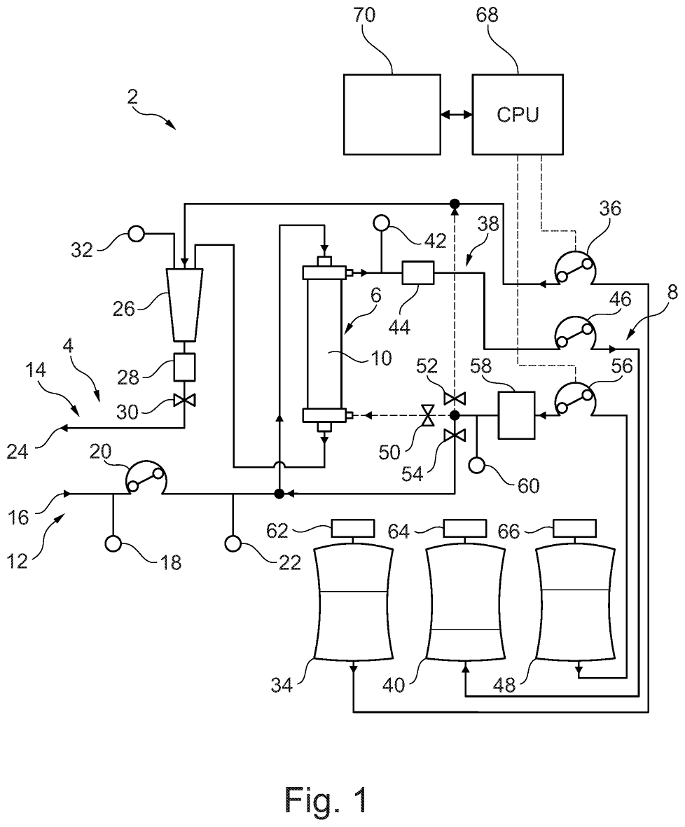

FIG. 1 shows a schematic view of an extracorporeal blood treatment device (dialysis device) 2. The blood treatment device 2 is basically configured to be used in both continuous and intermittent blood treatment therapies, in particular renal replacement therapies. The blood treatment device 2 is configured in particular as an acute dialysis machine or an acute dialysis device and is thus essentially prepared for use in intensive care units with predominantly unstable patients. With the blood treatment device 2 of the present disclosure, principally a variety of different blood treatment therapies can be performed (e.g. slow continuous ultrafiltration (SCUF), continuous veno-venous hemofiltration (CVVH), continuous veno-venous hemodialysis (CVVHD), continuous veno-venous hemodiafiltration (CVVHDF), therapeutic plasma exchange (TPE), etc.) as well as dilution modes (e.g., pre-dilution, post-dilution, pre-dilution and post-dilution) and anticoagulation types (e.g., none, heparin, citrate, etc.).

The blood treatment device 2 basically has an extracorporeal circuit 4, a dialyzer (hemofilter) 6 and a dialysis fluid circuit 8. The extracorporeal circuit 4 and the dialysis fluid circuit 8 are separated by a membrane 10 provided in the dialyzer 6, through which blood can be filtered using a dialysis fluid solution or without using a dialysis fluid solution.

The extracorporeal circuit 4 comprises an arterial portion 12 and a venous portion 14. In principle, it is provided that the arterial portion 12, in particular one end thereof, is to be connected or attached to an artery of a patient, in particular an intensive care patient. It is also provided that the venous portion 14, in particular one end thereof, is to be connected or attached to a vein of a patient, in particular an intensive care patient.

The arterial portion 12 has, starting from an arterial end 16 in a blood flow direction towards the dialyzer 6, an arterial pressure sensor 18, an (arterial) blood pump 20, and a dialyzer inlet pressure sensor 22. Starting from the dialyzer 6 in a blood flow direction towards a venous end 24, the venous portion 14 has a venous expansion chamber or air trap 26, a safety air detector 28 and a safety valve 30. A venous pressure can be measured on/behind the venous expansion chamber 26 using a venous pressure sensor 32.

As shown in FIG. 1, the venous expansion chamber 26 is connected to a substitution solution bag/container 34. A substitution solution pump 36 is provided and configured to pump a substitution solution from the substitution solution bag 34 into the extracorporeal blood circuit 4, in particular into the venous portion 14 thereof (into the venous expansion chamber 26).

The dialysis fluid circuit 8 has at least one outlet 38 for effluent/used dialysis fluid (dialysate)/another fluid. In principle, the effluent/dialysate/the other liquid can flow through the outlet 38 from the dialyzer 6 to a collecting bag/container 40 for effluent/dialysate/etc. In the outlet 38, an effluent pressure sensor 42, a blood leak detector 44 and an effluent pump 46 are arranged or provided in a direction of flow from the dialyzer 6 to the collecting bag 40.

As can be further seen in FIG. 1, a further bag/container 48 is provided in addition to the substitution solution bag 34 and the collecting bag 40. Depending on the desired blood treatment therapy to be performed, the bag 48 may contain, for example, a substitution solution/fluid or a dialysis fluid.

When, for example, a hemodialysis/hemodiafiltration treatment etc. is to be carried out with the extracorporeal blood treatment device 2, i.e. a blood treatment therapy in which dialysis fluid flows through the dialyzer 6 and thus a substance transport from the extracorporeal circuit 4 to the dialysis fluid circuit 8 takes place both by diffusion and convection, then the bag 48 contains dialysis fluid. When a first valve 50 is now opened and both a second valve 52 and a third valve 54 are closed, then the dialysis fluid can be pumped to the dialyzer 6 via a pump 56.

When, for example, hemofiltration etc. is to be performed with the extracorporeal blood treatment device 2, i.e. a blood treatment therapy in which no dialysis fluid flows through the dialyzer 6 and thus substance transport from the extracorporeal circuit 4 to the dialysis fluid circuit 8 takes place only via convection/filtration, the bag 48 can contain a substitution solution. When the first valve 50 and the second valve 52 are closed and the third valve 54 is opened, the substitution solution can be pumped from the bag 48 into the arterial portion 12 of the extracorporeal circuit 4 (pre-dilution). When the first valve 50 and the third valve 54 are closed and the second valve 52 is opened, the substitution solution can be pumped from the bag 48 into the venous portion 14 of the extracorporeal circuit 4 (post-dilution). When the first valve 50 is closed and the second valve 52 and the third valve 54 are opened, the substitution solution can be pumped from the bag 48 into both the arterial portion 12 and the venous portion 14 of the extracorporeal circuit (pre-dilution and post-dilution). According to the present disclosure, pre-dilution and post-dilution can also be achieved by pumping the substitution solution from the substitution solution bag 34 via the substitution solution pump 36 into the venous portion 14 of the extracorporeal circuit 4 (post-dilution) and simultaneously pumping the substitution solution from the bag 48 via the pump (substitution solution pump) 56 into the arterial portion 12 of the extracorporeal circuit 4 (pre-dilution).

As shown in FIG. 1, a fluid warmer 58 and a pressure sensor 60 are provided between the pump 56 and the valve assembly consisting of the first valve 50, the second valve 52, and the third valve 54.

The three bags, i.e. the substitution solution bag 34, the collecting bag 40 and the bag 48, each have load cells attached to them, namely a first load cell 62, a second load cell 64 and a third load cell 66. The first load cell 62 is basically configured to measure or monitor the weight of the substitution solution bag 34. The second load cell 64 is basically configured to measure or monitor the weight of the collecting bag 40. The third load cell 66 is basically configured to measure or monitor the weight of the bag 48.

The extracorporeal blood treatment device 2 furthermore has a control unit (CPU) 68, which receives information from the sensors provided in the blood treatment device 2 and which controls the actuators provided in the blood treatment device 2. According to the disclosure, this provides software-supported therapy in particular. The control unit 68 receives in particular information from the arterial pressure sensor 18, the dialyzer inlet pressure sensor 22, the safety air detector 28, the venous pressure sensor 32, the effluent pressure sensor 42, the blood leak detector 44, the pressure sensor 60, the first load cell 62, the second load cell 64, the third load cell 66, etc. The control unit 68 controls in particular the blood pump 20, the safety valve 30, the substitution solution pump 36, the effluent pump 46, the first valve 50, the second valve 52, the third valve 54, the pump 56, the fluid warmer 58, etc. Furthermore, the control unit 68 exchanges information with a user interface 70 designed as a display with touch screen. For example, the control unit 68 may be configured to display a warning or an alarm on the user interface 70. Furthermore, information entered by a user/operator on the user interface 70 can be transferred to the control unit 68.

As already shown in FIG. 1, the present disclosure essentially relates to the driving of the substitution solution pump 36 and the pump 56 (if the pump 56 works as a substitution solution pump). The present disclosure essentially relates to the control by the control unit 68. The control unit 68 can in particular calculate a difference or a backlog between an ideal/optimum target volume of the supplied substitution solution set by a user and an actually controlled volume of the supplied substitution solution. For this purpose, the control unit 68 uses a time curve of the flow rate of the substitution solution pump 36 or of the pump 56.

When the control unit 68 detects/when the control unit 68 becomes aware (by a corresponding calculation) that there is a difference or backlog between an ideal/optimum target volume set by a user and an actual/concretely controlled volume of the supplied substitution solution, the control unit 68 temporarily increases a controlled flow rate of the substitution solution pump 36 or of the pump 56 by a predetermined, fixed percentage. This means that the flow rate of the substitution solution pump 36 or the pump 56 is set to be higher than a normally required flow rate by a predetermined, fixed percentage. A normally required flow rate is understood to be a flow rate by means of which the ideal/optimum target volume set by a user could be achieved if there were no backlog/difference between the set target volume and the actually controlled volume of the supplied substitution solution.

The predetermined, fixed percentage can generally be set to a value between 1% and 5%. It may also be provided that the predetermined, fixed percentage is set higher if the deviation between the actual volume and the target volume is large, than if the deviation between the actual volume and the target volume is small. For example, the predetermined, fixed percentage can be set to 1% if the deviation is small and the predetermined, fixed percentage can be set to 5% if the deviation is large. In any case, the percentage set by the control unit (depending on the difference/backlog) is already preset and predetermined.

According to the disclosure, the flow rate/volume flow of the substitution solution pump 36 or of the pump 56 is increased by the predetermined, fixed percentage until the difference or the backlog between the actually controlled volume and the ideal target volume no longer exists, i.e. the actually controlled volume corresponds (again) to the ideal target volume.

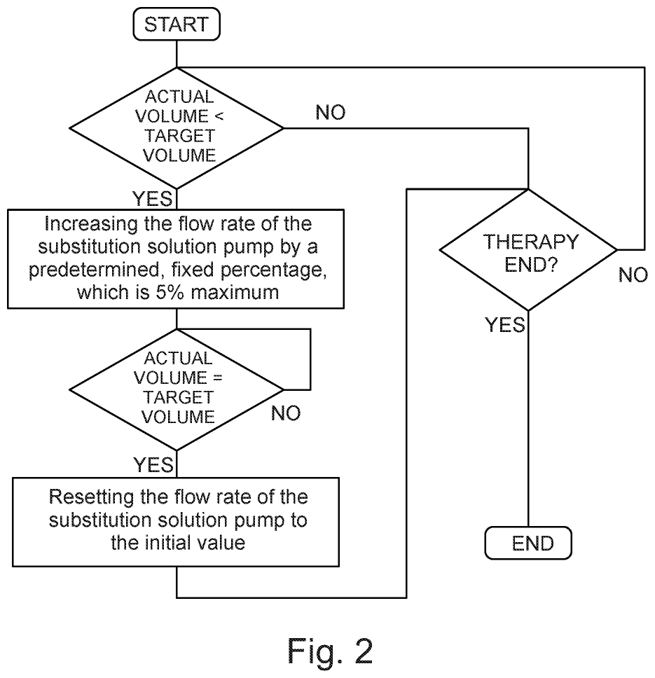

FIG. 2 shows the course of an automatic volume compensation of a substitution solution according to the disclosure. The control unit 68 first calculates an actually controlled volume of the substitution solution, which is supplied to an extracorporeal circuit 4. The control unit 68 then compares the actually controlled volume supplied to the extracorporeal circuit 4 with a (predetermined) ideal target volume. If the actually supplied volume or actual volume is smaller than the ideal target volume, the control unit increases the flow rate of a substitution solution pump by a predetermined, fixed percentage, which is at most 5%. Then the control unit 68 continues to compare the target volume with the actual volume. Only when the target volume is equal to the actual volume does the control unit 68 reset the flow rate of the substitution solution pump to the initial value/actually required value. The routine shown only ends when the therapy has ended.

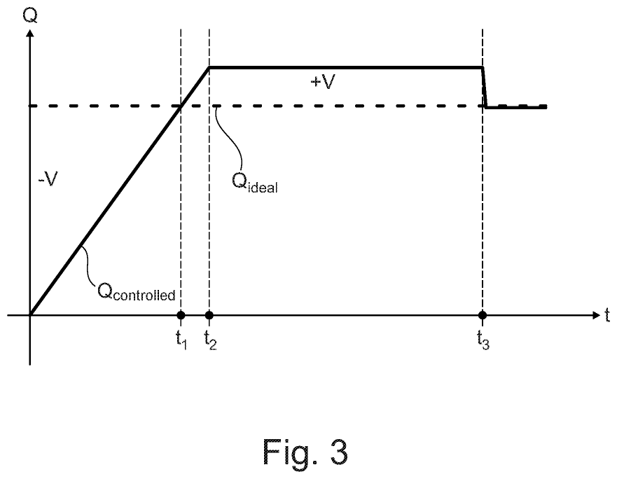

FIG. 3 shows a diagram showing the time course of a substitution solution flow rate Q_{controlled} of the substitution solution pump 36 or of the pump 56 controlled by the control unit 68. In particular FIG. 3 shows that when starting or restarting the substitution solution pump 36 or the pump 56, the substitution solution flow rate Q_{controlled} slowly/continuously/linearly increases (from zero) so that a desired ideal flow rate Q_{ideal} set by a user, which would result in the ideal/optimum target volume being supplied to the extracorporeal circuit 4 (if it was set/available from the start), is only reached at a time t1. According to the present disclosure, the controlled substitution solution flow rate Q_{controlled} is not (yet) set to the ideal flow rate Q_{ideal} set by the user at time t1, but continues to increase linearly until a controlled flow rate Q_{controlled} is reached, which is increased by a predetermined, fixed percentage compared to the ideal flow rate Q_{ideal}. This is the case in FIG. 3 at time t2. Now, the controlled flow rate is temporarily maintained at a constant value until the volume not yet supplied at the startup (see ‘−V’ in FIG. 3), i.e. the backlog or difference, has been completely compensated (see ‘+V’ in FIG. 3). This is the case in FIG. 3 at time t3. At time t3 the controlled flow rate Q_{controlled} is finally set to the ideal flow rate Q_{ideal}.

What is claimed:

1. A blood treatment device for use in blood treatment therapies, comprising:
an extracorporeal blood circuit, a dialyzer and a dialysis fluid circuit, wherein the extracorporeal blood circuit and the dialysis fluid circuit are separated from each other via a membrane provided in the dialyzer, via which blood can be filtered;
at least one substitution solution pump, which is configured to supply a substitution solution to the extracorporeal blood circuit before and/or after the dialyzer; and
a control unit which is configured to calculate a difference or a backlog between an ideal target volume set by a user and an actually controlled volume of the supplied substitution solution, and to temporarily increase a controlled flow rate of the substitution solution pump under corresponding controlling thereof by a predetermined, fixed percentage which is less than or equal to 5% until the actually controlled volume equals the ideal target volume.

2. The blood treatment device according to claim 1, wherein the predetermined, fixed percentage by which the flow rate of the substitution solution is increased is at least 1% and at most 5%.

3. The blood treatment device according to claim 1, wherein the predetermined, fixed percentage is set by the control unit depending on the missing volume, so that the predetermined, fixed percentage is set higher when the deviation between the actual volume and the target volume is large, than when the deviation between the actual volume and the target volume is small.

4. The blood treatment device according to claim 1, wherein the control unit is configured to increase the flow rate of the substitution solution pump only if other restrictions do not prohibit this.

5. The blood treatment device according to claim 1, wherein the control unit is configured to calculate the difference or the backlog between the ideal target volume and the actually controlled volume using the course of the flow rate of the at least one substitution solution pump.

6. The blood treatment device according to claim 1, wherein the control unit is configured to adjust the flow rate of the at least one substitution solution pump.

7. The blood treatment device according to claim 6, wherein, when starting or restarting the at least one substitution solution pump, the flow rate slowly increases so that a desired ideal flow rate is reached only after a predetermined, short time period.

8. The blood treatment device according to claim 7, wherein, after reaching the desired ideal flow rate, the flow rate is temporarily increased by the predetermined, fixed percentage in order to slowly reduce the difference or backlog between the actually controlled volume and the ideal target volume, which results from the slow increase of the flow rate at startup or restart, specifically until the actually controlled volume equals the ideal target volume.

9. The blood treatment device according to claim 1, wherein the control unit is configured so that, if the controlled flow rate of the substitution solution pump has to be temporarily reduced, the resulting backlog between the ideal target volume and the actually controlled volume is subsequently reduced or compensated for, by temporarily increasing the controlled flow rate of the substitution solution pump by the predetermined, fixed percentage under appropriate controlling thereof, specifically until the actually controlled volume equals the ideal target volume.
